(19) United States
(12) Patent Application Publication
(10) Pub. No.: US 2004/0005311 A1
(43) Pub. Date: Jan. 8, 2004

(54) Dietary supplement compositions

(76) Inventor: Bradford D. Pitman, Attleboro, MA (US)

Correspondence Address: LAHIVE & COCKFIELD, 28 STATE STREET, BOSTON, MA, 02109, US

(21) Appl. No.: 10/333,346

(22) PCT Filed: Jul. 20, 2001
(86) PCT No.: PCT/US01/22962

(51) Int. Cl. A61K 38/43; A61K 38/46; A61K 31/525; A61K 31/355; A61K 31/353; A61K 31/375; A61K 33/04; A61K 31/015; A61K 31/198

(52) U.S. Cl. 424/94.65; 514/458; 514/474; 424/702; 424/94.1; 514/564; 514/251; 514/456; 514/763

(57) ABSTRACT

The invention is directed to dietary supplements including a core of lysine and bromelain and, optionally, including lycopene. Supplements can further include at least one of Vitamin C, Vitamin E, Vitamin B12, CoEnzymeQ10, lycopene, folic acid, selenium, lecithin and quercetin. Supplements can also further include lutein or xanthin. A preferred supplement includes 5000 mg lysine, 125 mg bromelain, 250 mg Vitamin C,800 IU Vitamin E, 500 mcg Vitamin B 12, 200 mg CoEnzymeQ10, 10 mg lycopene, 800 mcg folic acid, 100 mcg selenium, 250 mg quercetin and 2400 mg lecithin. The supplements have dietary uses or can be used to alleviate dementia-related symptoms.

### RELATED APPLICATIONS

This application claims the benefit of prior-filed provisional patent application U.S. Serial No. 60/219,959, filed Jul. 20, 2000, the entire content of which is incorporated herein by this reference.

### BACKGROUND OF THE INVENTION

It is well known that the diets of many persons in today's modem societies are lacking in a variety of important or essential vitamins, minerals and other natural elements. From this knowledge has arisen a vast interest in dietary supplementation, to restore desirable or necessary levels of various vitamins, minerals and the like. Dietary supplementation has also become very popular as a natural approach to achieving improved health effects including weight loss, appetite suppression, increased energy levels, increased muscle mass, improved learning and memory and the like.

Moreover, it is well known that many disorders are the result of dietary deficiencies wherein the body is starved of certain vitamins, minerals and other natural elements. Other disorders are simply the result of aging. Disorders due to aging may result if the body produces too much or too little of certain enzymes or hormones, thereby affecting the body's metabolism. Some disorders can be treated or corrected by supplementing missing natural elements which are ordinarily not found in the average diet. Through the use of a daily supplement, supplying these missing vitamins and natural elements, the symptoms of various disorders may improve or disappear entirely.

### SUMMARY OF THE INVENTION

The present invention is directed to a-dietary supplement designed to enhance or increase levels of select vitamins, nutrients (e.g., essential nutrients), amino acids and/or antioxidants in a subject, e.g., a human subject, desiring or in need of such supplementation. In one embodiment, the invention features a supplement that includes a sufficient amount of lysine and bromelain. In another embodiment, the invention features a supplement that includes a sufficient amount of lycopene. In yet another embodiment, the invention features supplements as set forth herein that include, in addition to the core components described, at least one component selected from the group consisting of lysine, bromelain, Vitamin E, CoEnzymeQ10 (“COQ10”), lycopene, folic acid, selenium, lecithin, lutein, and xanthin (e.g., zeaxanthin). In yet another embodiment, the dietary supplement includes at least two, or alternatively, three, four, five, six, seven, eight, nine or ten of the following: Vitamin C, Vitamin E, Vitamin B12, CoEnzymeQ10, folic acid, selenium, lecithin, quercetin, lutein, and xanthin (e.g., zeaxanthin).

The dietary supplements of the instant invention can be used by any subject who desires or is in need of enhanced or increased levels of at least one of the vitamins, nutrients (e.g., essential nutrients), amino acids and/or antioxidants of the supplements. In one embodiment, the subject is a human desiring or in need of enhanced or increased levels of lysine and/or bromelain. In another embodiment, the subject is a human desiring or in need of enhanced or increased levels of lycopene. In another embodiment, the subject is a human desiring or in need of enhanced or increased levels at least one component selected from the group consisting of lysine, bromelain, Vitamin C, Vitamin E, Vitamin B12, CoEnzymeQ10 (“COQ10”), lycopene, folic acid, selenium, lecithin, quercetin, lutein, and xanthin (e.g., zeaxanthin). In another embodiment, the subject is a human desiring or in need of enhanced or increased levels at least two, or alternatively, three, four, five, six, seven, eight, nine, ten, eleven, twelve or thirteen of the following: lysine, bromelain, Vitamin C, Vitamin E, Vitamin B12, CoEnzymeQ10, lycopene, folic acid, selenium, lecithin, quercetin, lutein, and xanthin (e.g., zeaxanthin).

The dietary supplements of the instant invention can be used by a subject having at least one symptom selected from the group consisting of learning deficit, memory deficit or memory loss, loss or deficit of perception, cognitive deficit, poor cognitive function and dementia. In another embodiment, the subject is a human exhibiting symptoms of or afflicted by a non-Alzheimer's dementia. In yet another embodiment, the subject is a human afflicted by Alzheimer's disease and/or its attendant symptoms. In yet another embodiment, the invention features methods for reducing symptoms of a non-Alzheimer's dementia. In yet another embodiment, the invention features methods for reducing or alleviating at least one symptom selected from the group consisting of learning deficit, memory deficit or memory loss, loss or deficit of perception, cognitive deficit, poor cognitive function and dementia, the methods including the step of supplementing the subject's diet with at least one of the formulations or combinations described herein. In yet another embodiment, the invention features methods for reducing or alleviating the symptoms of Alzheimer's disease, the methods including the step of supplementing the subject's diet with at least one of the formulations or combinations described herein.

Further featured are oral delivery means, for example, gels, capsules powders and/or tablets (e.g., formulated as described herein) as well as regimes for accomplishing the desired dietary supplementation and kits for accomplishing the same.

### DETAILED DESCRIPTION OF THE INVENTION

The present invention relates to novel combination or formulations for supplementing the diet of a subject desiring or in need of such dietary supplementation.

In one embodiment, the supplement comprises a “core formulation” which serves as the base or root of a more complex dietary supplement including at least one additional compound, agent or component. An exemplary “core formulation” includes a sufficient amount lysine and bromelain. Another exemplary “core formulation” includes a sufficient amount lysine, bromelain and lycopene. In one embodiment, the dietary supplement comprises the core formulation (e.g., lysine and bromelain) and at least one agent selected from the group consisting of Vitamin C, Vitamin E, Vitamin B12, CoEnzymeQ10, lycopene, folic acid, selenium, lecithin, quercetin, lutein, and xanthin (e.g., zeaxanthin). In another embodiment, the dietary supplement includes a core formulation (e.g., lysine, bromelain and lycopene) and at least one agent selected from the group consisting of Vitamin C, Vitamin E, Vitamin B12, CoEnzymeQ10, lycopene, folic acid, selenium, lecithin, quercetin, lutein, and xanthin (e.g., zeaxanthin). A preferred dietary supplement includes a core formulation (e.g., lysine and bromelain) and at least lycopene, CoEnzymeQ10, lecithin, Vitamin E, selenium, folic acid and, optionally, a multivitamin (see e.g., Example 1).

In another embodiment, the invention features a dietary supplement including CoEnzymeQ10, a choline source and at least one antioxidant. As used herein, a “choline source” includes a vitamin or vitamin-like nutrient that is utilized by cells to generate choline. Preferred “choline sources” include, but are not limited to lecithin, optionally in combination with lysine. An “antioxidant”, as used herein, includes a vitamin or vitamin-like nutrient that quenches or neutralizes free radicals in the body, limiting cellular damage. Exemplary antioxidants include but are not limited to Vitamins E and C, alpha carotene, beta carotene, lutein, zeaxanthin, lycopene and selenium.

In yet another embodiment, the invention features a dietary supplement including lycopene (e.g., a sufficient amount of lycopene). In another embodiment, the invention features a dietary supplement including lycopene (e.g., a sufficient amount of lycopene) and at least one agent selected from the group consisting of lysine, bromelain, Vitamin C, Vitamin E, Vitamin B12, CoEnzymeQ10, folic acid, selenium, lecithin, quercetin, lutein, and xanthin (e.g., zeaxanthin). Preferably, a dietary supplement includes sufficient amounts of lycopene and lysine.

In another embodiment, the dietary supplement includes lycopene, lysine, and at least one agent selected from the group consisting bromelain, Vitamin C, Vitamin E, Vitamin B12, CoEnzymeQ10, lycopene, folic acid, selenium, lecithin, quercetin, lutein, and xanthin (e.g., zeaxanthin). In yet another embodiment, the dietary supplement includes the agents described herein as Formula A, Formula B, Formula C (e.g., Formula C1 or C2) or Formula D (e.g., Formula D1 or D2). Optionally, any one of Formulas A, B, C or D can additionally include a sufficient amount of lutein or xanthin (e.g., zeaxanthin). A subject can take Formula A, Formula B or Formula C but, preferably takes Formula A, Formula B and Formula C sequentially. Alternatively, a subject can take Formula A, Formula B, Formula C and Formula D sequentially, or can take only a supplement of Formula D.

In yet another embodiment, the invention features a dietary supplement combining agents selected from the group consisting of: lysine, bromelain, Vitamin C, Vitamin E, Vitamin B12, CoEnzymeQ10, lycopene, folic acid, selenium, lecithin, quercetin, lutein, and xanthin (e.g., zeaxanthin). In a preferred embodiment, the supplement includes at least any two, or alternatively any three, four, five, six, seven, eight, nine, ten, eleven, twelve or thirteen of the following agents or components: lysine, bromelain, Vitamin C, Vitamin E, Vitamin B12, CoEnzymeQ10, lycopene, folic acid, selenium, lecithin, quercetin, lutein, and xanthin (e.g., zeaxanthin). As defined herein, a “sufficient amount” is an amount of a component included in the supplement such that when delivered to the subject, e.g., ingested by the subject, an increase or enhancement of the physiological levels of said component is detected or detectable. For example, a “sufficient amount” of a vitamin, mineral or other natural component is such that when formulated into the dietary supplement and delivered to the subject, an increase or enhancement of the level of vitamin, mineral or natural component is detected or detectable in the subjects bloodstream, cells, tissues, serum, urine, other biological fluid, etc.

Table 1 sets forth preferred amounts (e.g., sufficient amounts or effective amounts) of compounds, agents or components that are to be formulated into the dietary supplements of the present invention.

TABLE 1
(daily amounts)

| Agent/Component | Range of amount^{‡} | | Preferred amount | |
|---|---|---|---|---|
| lysine | 500-7500 | mg | 2000 | mg or |
| | | | 5000 | mg |
| bromelain | 50-1000 | mg | 125 | mg |
| Vitamin C | 50-500 | mg | 250 | mg |
| Vitamin E | 1000-2000 | IU | 800 | IU |
| Vitamin B12 | 50-1000 | mcg | 500 | mcg |
| CoEnzymeQ10 | 50-1000 | mg | 200 | mg |
| lycopene | 1-100 | mg | 10 | mg |
| folic acid | 400-2500 | mcg* | 8000 | mcg or |
| | | | 1200 | mcg |
| selenium | 50-600 | mcg | 100 | mcg |
| quercetin | 50-1000 | mg | 250 | mg |
| lecithin | 500-5000 | mg | 1200 | mg or |
| | | | 2400 | mg |
| lutein | 0.001-1 | g | 15 | mg |
| xanthin (e.g., | 0.001-1 | g | 12 | mg |
| zeaxanthin) | | | | |

^{‡}Amounts intermediate are also intended to be included.
*micrograms (i.e., μg)

Formulations and Preparations

For convenience in providing preferred amounts of the components listed above, multi-component dosages (e.g., powders, tablets, capsules or gels) are featured as part of the present invention. As used herein, a “multi-component dosage” includes a single dosage (e.g., a single aliquot of powder, single tablet, single capsule, single aliquot of gel) that includes at least two components and, optionally, includes a physiologically acceptable carrier. It can be readily appreciated that a subject may be more amenable to taking a fewer number of multi-component dosages (e.g., tablets, capsules, powder or gel aliquots) than, for example, the numerous single component dosages (e.g., pills) set forth in Example 1, “Daily Procedure”.

Preferred Multi-dosage Formulations

| | Per dosage | | Daily dose | |
|---|---|---|---|---|
| Formula A | | | | |
| Vitamin E | 134 | IU | 804 | IU |
| CoEnzymeQ10 | 33.3 | mg | 199.8 | mg |
| Lycopene | 1.67 | mg | 10.02 | mg |
| Folic Acid | 134 | mcg | 804 | mcg |
| Formula B | | | | |
| Vitamin E | | | 800 | IU |
| EnzymeQ10 | | | 200 | mg |
| Lycopene | | | 10 | mg |
| Folic Acid | | | 800 | mcg |
| Selenium | | | 100 | mcg |
| Bromelain | | | 125 | mg |
| Formulas C | | | | |
| Formula C1 | | | | |
| Vitamin E | | | 800 | IU |
| CoEnzymeQ10 | | | 200 | mg |
| Lycopene | | | 10 | mg |
| Folic Acid | | | 800 | mcg |
| Bromelain | | | 125 | mg |
| Lysine | | | 2000 | mg |
| Lecithin | | | 1200 | mg |
| Formula C2 | | | | |
| Vitamin E | | | 800 | IU |
| CoEnzymeQ10 | | | 200 | mg |
| Lycopene | | | 10 | mg |
| Folic Acid | | | 800 | mcg |
| Bromelain | | | 125 | mg |
| Lysine | | | 5000 | mg |
| Lecithin | | | 2400 | mg |
| Formulas D | | | | |
| Formula D1 | | | | |
| Vitamin C | | | 250 | mg |
| Vitamin E | | | 800 | IU |
| Vitamin B12 | | | 500 | mcg |
| CoEnzymeQ10 | | | 200 | mg |
| Lycopene | | | 10 | mg |
| Folic Acid | | | 800 | mcg |
| Bromelain | | | 125 | mg |
| Lysine | | | 2000 | mg |
| Lecithin | | | 1200 | mg |
| Selenium | | | 100 | mcg |
| Quercetin | | | 250 | mg |
| Formula D2 | | | | |
| Vitamin C | | | 250 | mg |
| Vitamin E | | | 800 | IU |
| Vitamin B12 | | | 500 | mcg |
| CoEnzymeQ10 | | | 200 | mg |
| Lycopene | | | 10 | mg |
| Folic Acid | | | 800 | mcg |
| Bromelain | | | 125 | mg |
| Lysine | | | 5000 | mg |
| Lecithin | | | 2400 | mg |
| Selenium | | | 100 | mcg |
| Quercetin | | | 250 | mg |

Formula E

Any of the above Formulations A, B, C or D, further including xanthin (e.g., zeaxanthin) and/or lutein. Liquid formulations are also within the scope of the invention. A preferred liquid formulation and dosing comprises for example, one measured aliquot (e.g., teaspoon) including the quantities of components in any of the above formulas, preferably formula D2, taken 1, 2 or 3 times a day, preferably three times a day. Alternatively, dosing can comprise taking 1, 2 or 3 times the above-described quantities of components in a single dosage unit, for example, in a single tablespoon of a liquid formulation.

In a preferred embodiment, the supplements of the present invention optionally include at least one physiologically acceptable carrier. The phrase “physiologically acceptable carrier” as used herein means a physiologically acceptable material, composition or vehicle, such as a liquid or solid filler, diluent, excipient, solvent or encapsulating material, involved in carrying or transporting compound(s), agent(s), component(s) or formulation(s) of the present invention within or to the subject such that they can performs their intended function. Each carrier must be “acceptable” in the sense of being compatible with the other components (e.g., therapeutically effective components) of the formulation and not injurious to the subject. Some examples of materials which can serve as physiologically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in physiological formulations.

Wetting agents, emulsifiers and lubricants, such as sodium lauryl sulfate and magnesium stearate, as well as coloring agents, release agents, coating agents, sweetening, flavoring, perfuming agents and preservatives can also be present in the formulations.

Methods of preparing these formulations or compositions include the step of bringing into association an active component of the present invention with the carrier and, optionally, one or more accessory ingredients. In general, the formulations are prepared by uniformly and intimately bringing into association an active component of the present invention with liquid carriers, or finely divided solid carriers, or both, and then, if necessary, shaping the product.

Formulations of the invention suitable for oral administration (i.e., oral delivery means) may be in the form of gels, capsules, cachets, pills, tablets, lozenges, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a therapeutically effective component of the present invention as an active ingredient. A formulation of the present invention may also be administered as a paste.

In solid dosage forms of the invention for oral administration (capsules, tablets, pills, powders and the like), the active ingredient is mixed with one or more physiologically acceptable carriers, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets, pills and powders, the therapeutic formulations may also comprise buffering agents. Solid formulations of a similar type may also be employed as fillers in soft and hard-filled gelatin capsules using such excipients as lactose or milk sugars, as well as high molecular weight polyethylene glycols and the like.

A tablet may be made by compression or molding powdered components, optionally with one or more accessory ingredients. Compressed tablets may be prepared using at least one binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered component moistened with an inert liquid diluent.

The tablets, and other solid dosage forms of the therapeutic formulations of the present invention, such as capsules, powders and/or pills, may optionally be scored or prepared with coatings and shells, such as enteric coatings and other coatings well known in the relevant art. They may also be formulated so as to provide slow or controlled release of the active ingredient therein using, for example, hydroxypropylmethyl cellulose in varying proportions to provide the desired release profile, other polymer matrices, liposomes and/or microspheres. These formulations may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active component(s) can also be in micro-encapsulated form, if appropriate, with one or more of the above-described excipients.

Liquid dosage forms for oral administration of the formulations of the invention include physiologically acceptable emulsions, microemulsions, solutions, suspensions, syrups and elixirs. In addition to the active components, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral formulations can also include adjuvants such as wetting agents, emulsifying and suspending agents, sweetening, flavoring, coloring, perfuming and preservative agents.

Suspensions, in addition to the active components, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Actual levels of components of the invention may be varied so as to obtain amounts of individual active components which are effective to achieve the desired response for a particular subject, without being toxic to the subject. The amount of individual components or total dosage level may depend upon a variety of factors including the age, sex, weight, condition, general health and prior medical history of the subject taking the supplement, and like factors well known in the medical arts.

Therapeutic Uses

When used to achieve a desired therapeutically effect, e.g., to reduce or alleviate a symptom selected from the group consisting of learning deficit, memory deficit or memory loss, loss or deficit of perception, cognitive deficit, poor cognitive function and dementia, or to reduce, lessen, halt, or reverse at least one of the symptoms of Alzheimer's disease or a non-Alzheimer's dementia, the supplement can be formulated to include a therapeutically effective amount of one or more components of the supplement. As defined herein, a “therapeutically effective” amount includes an amount of an agent or component capable of producing the desired therapeutic effect (e.g., reducing or alleviating a symptom selected from the group consisting of learning deficit, memory deficit or memory loss, loss or deficit of perception, cognitive deficit, poor cognitive function and dementia, or reducing, lessening, halting, reversing at least one of the symptoms of Alzheimer's disease or a non-Alzheimer's dementia). It will be readily appreciated by one skilled in the art that when administered as a component of a formulation that includes more that one therapeutic or symptom-alleviating compound or agent, the amount of component necessary to achieve lessened or diminished symptoms may be a smaller or reduced amount than that required to produce a therapeutically effect when administered singly. Moreover, one skilled in the art will readily appreciate that the amount of a compound or agent necessary to produce a therapeutic effect (e.g., reduce one or more symptoms related to Alzheimer's disease or a non-Alzheimer's dementia) in a first patient or subject may vary significantly from that needed or required to produce a similar therapeutic effect in a second subject or patient. Accordingly, it is within the scope of the invention to vary the amounts of certain compounds, agents or components, for example, within a therapeutic formulation as defined herein. Ranges of acceptable therapeutically effective amounts can be selected from those set forth in Table I.

Preferably, a therapeutically effective amount of a compound, agent, component or formulation of the present invention is an amount sufficient to lessen, diminish, alleviate or reduce more that one Alzheimer's disease-related or Alzheimer's disease-associated symptom or non-Alzheimer's dementia-related or non-Alzheimer's disease-associated symptom, as described herein. Even more preferably, a therapeutically effective amount of a compound, agent, component or formulation of the present invention is an amount sufficient to slow the progression of Alzheimer's disease or a non-Alzheimer's dementia. Even more preferably, a therapeutically effective amount of a compound, agent, component or formulation of the present invention is an amount sufficient to stop the progression of Alzheimer's disease or a non-Alzheimer's dementia. Most preferably, a therapeutically-effective amount of a compound, agent, component or formulation of the present invention is an amount sufficient to reverse Alzheimer's disease or a non-Alzheimer's dementia, or the symptoms thereof, in a subject or patient.

When using the dietary supplements of the invention to reduce or alleviate a symptom or symptoms described above, the supplement can be formulated to further include (or the subject can further supplement their diet with) at least one agent or component selected from the group consisting of Vitamin B12, cranberry extract (e.g., about 810 mg daily, for prevention of urinary tract infections common to some patients), a daily multivitamin, and a daily aspirin (e.g., an enteric coated aspirin). Moreover, a subject's level of a preferred agent or component can be effectively increased by inclusion in the diet of said subject at least one foodstuff selected from the group consisting of a dietary antioxidant (e.g., grape juice, raisins, cantaloupe or another source of β-carotene), pineapple (a natural source of bromelain), tomato sauce products (a natural source of lycopene) and yellow corn (a natural source of at least lycopene, lysine, lutein and/or xanthin).

As used herein, the term “dementia” includes a form of brain failure in a subject or patient characterized by dysfunction or loss of cognitive function, physical function or behavioral function. The cause of brain failure can be genetic or environmental and results, at least in part, in a physically detectable dementia or dementia-associated symptom or syndrome exhibited by the subject or patient. Exemplary causes of dementia include, but are not limited to, structural causes, for example, Alzheimer's disease (AD), vascular disease, multi-infarct dementia, Binswanger's dementia, Huntington's cholera, multiple sclerosis, Pick's disease, cerebellar degeneration, Parkinson's disease, Wilson's disease, amyotrophic lateral sclerosis (ALS) , progressive multifocal leuko-encephalopathy, progressive supranuclear palsy, brain tumor, irradiation to frontal lobes, surgery, normal-pressure hydrocephalus, brain trauma, chronic subdural hematoma and dementia pugilistica; infectious causes, for example, neurosyphilis, tuberculosis and fungal meningitis, viral encephalitis, human immunodeficiency virus (HIV)-related disorders, prion diseases (e.g., Creutzfeldt-Jakob disease) and Gerstmann Stäussler syndrome; and metabolic-toxic causes, for example, anorexia, pernicious anemia, pellagra, folic acid deficiency, hypothyroidism, bromide intoxication, hypoglycemia, hypercalcemia associated with hyperparathyroidism, organ system failure, hepatic encephalopathy, respiratory encephalopathy, and chronic drug-alcohol-nutritional abuse; and the like. As used herein, the “non-Alzheimer's dementias” include multi-infarct dementia, Binswanger's dementia or subcortical arteriosclerotic encephalopathy, the dementia associated with Parkinson's disease and Huntington's disease, Pick's disease, the frontal lobe dementia syndromes, normal-pressure hydrocephalus, subdural hematoma, progressive multifocal leukoencephalopathy, the dementia associated with progressive supranuclear palsy, and the dementias associated with infections (e.g., Creutzfeldt-Jakob disease, Gerstmann-Sträussler's syndrome, and the acquired immunodeficiency syndrome).

As used herein, the phrases “dementia-associated symptoms”, “symptoms related to” or “symptoms associated with dementia”, for example, “Alzheimer's disease-related or “Alzheimer's disease-associated” or “non-Alzheimer's dementia-related” or “non-Alzheimer's dementia-associated” symptoms include physiological characteristics, for example, observable by a caregiver or detectable or diagnosed by a physician, that are exhibited by a patient or subject having dementia (e.g., Alzheimer's disease or a non-Alzheimer's dementia), or at risk of developing or in the early stages of development of dementia (e.g., Alzheimer's disease or a non-Alzheimer's dementia). Exemplary dementia-related symptoms include, but are not limited to, loss of weight, decreased or worsened mental status, confusion, difficulty following instructions, argumentative, combative, loss of short and/or long term memory, irregular sleep behavior or patterns (e.g., inability to stay awake during daytime hours or inability to sleep through the night), loss of logic, loss of alertness, incontinence, and the like.

The invention flier features methodologies (e.g., therapeutic methodologies) including administering to a patient or subject having Alzheimer's disease or a non-Alzheimer's dementia and/or Alzheimer's disease-related or non-Alzheimer's dementia-related symptoms, a dietary supplement, as described herein, or at least a therapeutically-effective amount of an active component of a dietary supplement described herein.

When used as part of a therapy, it is understood that a caregiver can determine or alter the amount or dosages of therapeutic formulation to be administered. For example, the caregiver can start with dosages or daily doses of the formulations of the invention at levels lower than that recommended in Table I and gradually increase the dosages or daily dose until the desired effect is achieved. In general, a suitable daily dose of a formulation of the invention will be that amount of the formulation which is the lowest dose effective to produce the desired therapeutic effect. Such an effective dose will generally depend upon the factors described above. As described herein, the total effective daily dose of a formulation of the present invention may be administered as two, three, four, five, six or more sub-doses administered separately at appropriate intervals throughout the day, optionally, in unit dosage forms.

A preferred dosage regimen includes: the recommended daily dose of the formulation of Formula A administered, for example, in 1, 2, 3, 4, 5, 6, or more unit dosages, for about 16 days; followed by the recommended daily dose of the formulation of Formula B administered, for example, in 1, 2, 3, 4, 5, 6, or more unit dosages, for about 20 days; followed by the recommended daily dose of the formulation of Formula C (e.g., Formula C1 or C2) administered, for example, in 1, 2, 3, 4, 5, 6, or more unit dosages, for about 30 days. Subsequent to the above-described exemplary dosage regime, a subject or patient can be kept on a maintenance program of, for example, the recommended daily dose of the formulation of Formula C or Formula D (e.g., Formula D1 or D2).

The present invention also pertains to packaged therapeutic formulations. The packaged therapeutic formulations include a container holding, for example, daily dosages compartmentalized by days of the week, of at least one formulation as described supra and instructions for use, for example, instructions for using the formulations for reducing or alleviating at least one symptom selected from the group consisting of learning deficit, memory deficit or memory loss, loss or deficit of perception, cognitive deficit, poor cognitive function and dementia, or for treating and/or reducing symptoms of Alzheimer's disease or a non-Alzheimer's dementia (e.g., Alzheimer's Disease and related dementia) in the subject. In the case of powder formulations, a packaged formulation can include a measuring means, for example, a spoon or scoop, designed to measure a single dosage unit. Alternatively, a single dosage unit of a powder formulation can be packaged in a packet, for example a paper or plastic packet. Packaged therapeutic formulations of the present invention can further include, for example, a calendar with directions as to daily doses to be administered.

Exemplification

### EXAMPLE 1

Example 1 sets forth an account of the supplementation of the diet of a first dementia subject which led, at least in part, to the formulation of the preferred supplements of the present invention.

Prior to addressing the patient's dementia problem, a complete diagnosis of the subject was performed by a qualified physician. The subject was described as having fairly severe symptom's of Alzheimer's disease, having lost weight and exhibiting a marked worsening in mental status (as compared to previous visits with the same physician). The subject had become increasingly sleepy with poor concentration and was noted to be increasingly confused. She had difficulty following instructions and was incontinent at the time of the examination. She was combative and her family was aware of her altered condition and had been advised to place her in a geriatric psychiatric unit in order to alleviate the burden of caring for her at home.

The treatment process of this subject developed based on an initial observation by the subject's caregiver that feeding the subject corn (later identified as a dietary source of lysine), resulted in a marked change in the subject's condition from virtually non-functional to functional. This caused the caregiver (i.e., the present inventor) to begin adding components to the subject's diet including both dietary components as well as supplements (e.g., dietary supplements). Through a system of trial and process-of-elimination, the caregiver settled on the following daily procedure of food and dietary supplements and witnessed the subject's dramatic improvement in both physical and mental condition.

Daily procedure

| Mornings | | | |
|---|---|---|---|
| 250 | mg Vitamin C | 81 | mg enteric coated aspirin |
| 500 | mcg Vitamin B12 | 250 | mg quercetin |
| 125 | mg Bromelain | 1000 | mg lysine |
| 1200 | mg lecithin | 810 | mg cranberry extract |
| 100 | mg Ultra CoQ10 | 400 | IU Vitamin E |
| 400 | mcg folic acid | | |
| Daily vitamin containing | | | |
| | Vitamin A | 5000 | IU (5% beta carotene) |
| | Vitamin C | 60 | mg |
| | Vitamin D | 400 | IU |
| | Vitamin E | 30 | IU |
| | thiamin | 1.5 | mg |
| | riboflavin | 1.7 | mg |
| | niacin | 20 | mg |
| | Vitamin B6 | 2 | mg |
| | folate | 400 | mcg |
| | Vitamin B12 | 6 | mcg |
| | pantothenic acid | 10 | mg |

| Afternoon | |
|---|---|
| 1000 | mg lysine |
| 810 | mg cranberry extract |
| 5 | mg lycopene |
| Evening | |
| 1000 | mg lysine |
| 1200 | mg lecithin |
| 810 | mg cranberry extract |
| 100 | mg Ultra CoQ10 |
| Night | |
| 1000 | mg lysine |
| 400 | IU Vitamin E |
| Night (middle of night) | |
| 1000 | mg lysine |
| Every seven days | |
| 200 | mcg selenium |

In summary, the subject's treatment consisted of the following:

Subject #1

5000 mg lysine daily

+5 mg lycopene daily

+CoQ10 200 mg daily

+Bromelain 125 mg daily

+2400 mg lecithin daily

+800 mcg folic acid daily

+250 mg quercetin daily

+2430 mg cranberry extract daily

+800 IU Vitamin E daily

+a daily multivitamin (no zinc)

+a daily aspirin

+500 mcg B12

+81 mg enteric coated aspirin

+250 mg Vitamin C

+200 mcg selenium once every 7 days

The caregiver also found it beneficial to minimize or eliminate certain things from the subject's diet including red meats (exemplary of a difficult to digest foodstuff), white flour-based foodstuffs and potatoes (exemplary of low nutritional value foodstuffs) and zinc. The latter was excluded from the subject's diet, in particular, because she had been diagnosed as having macular degeneration and about six to eight months prior to the first appearance of Alzheimer's disease-related symptoms, had begun taking Occuvite™, a vitamin having 267% of the daily requirement of zinc, in addition to a daily multivitamin having 100% of the daily recommended zinc. The caregiver noted the coincidence between high dietary zinc and the appearance of Alzheimer's-related symptomology. For this reason, a multivitamin without zinc is also recommended.

Approximately two years after the physician's first account of the subject's condition set forth above, the physician described the subject as “markedly improved, alert, able to concentrate, able to tell a joke, answering questions well, previously she had been unable to answer questions, with good short term memory and logic”.

The patient remained almost completely normal and thus enjoyed, from the time shortly following her initial treatment until her death (ultimately dying of heart complications) approximately three additional years of her life: a decent life, with her family and out of a nursing home.

### EXAMPLE 2

Example 2 describes a second subject receiving a similar treatment to that set forth in Example 1.

Prior to placing the subject on the following daily procedure, she was argumentative and confused (e.g., lost the ability to tell time as well as exhibiting diminished concept of time periods), had significant loss of short term memory, had difficulty remembering names and finding nouns to describe what she saw or thought, had confused long term memory (e.g., putting together different times of her life and telling it as one memory. The subject's physician described her as having a “dementia-type disease”. The subject was initially placed on the prescription drug, Aricept but was observed to become more confused.

Upon initially including in her diet significant quantities of corn, tomato sauce products and pineapple and supplementing these foodstuffs with Vitamin E, Vitamin B-12 and lysine, the subject became more focused and participated in conversations more. In subsequent weeks, her logical thinking improved, she was better able to initiate and participate in conversations, had less difficulty finding the appropriate words to use and demonstrated improved logical thinking (although her concept of time did not seem to improve). The subject was more alert and her short-term memory improved. With continued treatment, her long-term memory also improved (e.g., she was able to joyously tell stories of people and events of the past).

In summary, the subject's treatment consisted of the following:

Subject #2

500 mg lysine (3 times daily)

+1000 mg B12

+800 IU Vitamin E (400 IU and the AM and 400 IU in the PM)

(+pineapple juice)

### EXAMPLE 3

Example 3 summarizes data collected from 4 additional subjects who complied with the therapeutic regimes described in Examples 1 and 2 to varying degrees.

Subject #3 initially exhibited symptoms including memory loss and anxiety and was noticed to be eating less. The subject began taking lysine, lycopene, folic acid, Vitamin E and a daily multivitamin and showed improvement. The subject eventually reduced therapy to taking lycopene only (e.g., 45 mg lycopene daily) or, periodically, lycopene and lysine, still exhibiting reduced symptoms.

Subject #4 exhibited Alzheimer's-related symptoms including an inability to eat and incontinence and showed marked improvement after starting a regime of lysine, lycopene, CoEnzymeQ10, Vitamin E and a daily multivitamin.

Subject #5 showed marked improvement after starting a regime of lysine, lycopene, folic acid and Vitamin E.

Subject #6 showed improvement following a regime of CoEnzymeQ10, lysine, lycopene, folic acid and a daily multivitamin.

The diets of these four subjects were also supplemented, to varying degrees, with yellow corn, pineapple and tomato sauce products.

Conclusion

The dramatic change observed in subjects #1 and #2 has been reproduced in at least four other subjects to a degree in direct relation to their commitment and consistency in following the therapeutic regimes described herein.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A dietary supplement comprising between 500-7500 mg lysine and 50-1000 mg bromelain.

2. A dietary supplement comprising about 2000 mg lysine and about 125 mg bromelain.

3. A dietary supplement comprising about 5000 mg lysine and about 125 mg bromelain.

4. The dietary supplement of any of claims 1 to 3, further comprising between 1-100 mg lycopene.

5. The dietary supplement of any of claims 1 to 3, further comprising 10 mg lycopene.

6. The dietary supplement of any one of claims 1 to 5, further comprising a sufficient amount of at least one component selected from the group consisting of Vitamin E, CoEnzymeQ10, lycopene, folic acid, selenium, lecithin and quercetin.

7. The dietary supplement of claim 6, comprising between 50-500 mg Vitamin C.

8. The dietary supplement of claim 6, comprising about 250 mg Vitamin C.

9. The dietary supplement of claim 6, comprising between 1000-2000 IU Vitamin E.

10. The dietary supplement of claim 6, comprising about 800 IU Vitamin E.

11. The dietary supplement of claim 6, comprising between 50-1000 mcg vitamin B12.

12. The dietary supplement of claim 6, comprising about 500 mcg Vitamin B12.

13. The dietary supplement of claim 6, comprising between 50-1000 mg CoEnzymeQ10.

14. The dietary supplement of claim 6, comprising about 200 mg CoEnzymeQ10.

15. The dietary supplement of claim 6, comprising between 1-100 mg lycopene.

16. The dietary supplement of claim 6, comprising about 10 mg lycopene.

17. The dietary supplement of claim 6, comprising between 400-2500 mcg folic acid.

18. The dietary supplement of claim 6, comprising about 1200 mcg folic acid

19. The dietary supplement of claim 6, comprising between 50-600 mcg selenium.

20. The dietary supplement of claim 6, comprising about 200 mcg selenium.

21. The dietary supplement of claim 6, comprising between 500-5000 mg lecithin.

22. The dietary supplement of claim 6, comprising about 1200 mg lecithin.

23. The dietary supplement of claim 6, comprising about 2400 mg lecithin.

24. The dietary supplement of claim 6, comprising between 50-1000 mg quercetin.

25. The dietary supplement of claim 6, comprising about 250 mg quercetin.

26. The dietary supplement of any one of the preceding claims, further comprising a sufficient amount of lutein or xanthin.

27. The dietary supplement of claim 26, comprising between 0.001-1 gram lutein.

28. The dietary supplement of claim 26, comprising about 15 mg lutein.

29. The dietary supplement of claim 26, comprising between 0.001-1 gram xanthin.

30. The dietary supplement of claim 26, comprising about 12 mg xanthin.

31. A dietary supplement comprising Formula A.

32. A dietary supplement comprising Formula B.

33. A dietary supplement comprising Formula C1 or C2.

34. A dietary supplement comprising between 500-7500 mg lysine, between 50-1000 mg bromelain, between 50-500 mg Vitamin C, between 1000-2000 IU Vitamin E, between 50-1000 mcg Vitamin B12, between 50-1000 mg CoEnzymeQ10, between 1-100 mg lycopene, between 400-2500 mcg folic acid, between 50-600 mcg selenium, between 50-1000 mg quercetin, and between 500-5000 mg lecithin.

35. A dietary supplement comprising 5000 mg lysine, 125 mg bromelain, 250 mg Vitamin C, 800 IU Vitamin E, 500 mcg Vitamin B12, 200 mg CoEnzymeQ10, 10 mg lycopene, 800 mcg folic acid, 100 mcg selenium, 250 mg quercetin and 2400 mg lecithin.

36. A dietary supplement comprising Formula D1 or D2.

37. The dietary supplement of any one of claims 31 to 35, further comprising lutein or xanthin.

38. A method for reducing or alleviating at least one symptom selected form the group consisting of learning deficit, memory deficit or memory loss, loss or deficit of perception, cognitive deficit, poor cognitive function and dementia, said method comprising the step of supplementing the diet of a subject exhibiting said symptom with the dietary supplement of any one of claims 1-37, thereby reducing or alleviating said symptom.

39. A method of reducing symptoms of dementia, said method comprising the step of supplementing the diet of a subject exhibiting said symptom with the dietary supplement of any one of claims 1-37, thereby reducing said symptom.

40. The method of claim 39, wherein said dementia is Alzheimer's disease.

41. The method of claim 39, wherein said dementia is a non-Alzheimer's dementia.

42. The method of claim 38 or 39, wherein said method further comprises supplementing a diet of said subject with at least one foodstuff selected from the group consisting of a dietary antioxidant, pineapple, tomato sauce products and yellow corn.

43. The method of claim 42, wherein the dietary antioxidant is a caratenoid.

44. The method of claim 42, wherein the dietary antioxidant is selected from the group consisting of grape juice, raisins, cantaloupe.

45. The method of claim 38 or 39, wherein said method further comprises supplementing the subject's diet with at least one component selected from the group consisting of Vitamin B12, cranberry extract, a daily multivitamin, and a daily aspirin.

46. A method of reducing symptoms of Alzheimer's disease, said method comprising the step of supplementing the diet of a subject exhibiting the symptom with the dietary supplement of any one of claims 1-37, thereby reducing said symptom.

47. A method of reducing symptoms of Alzheimer's disease, said method comprising the step of supplementing the diet of a subject exhibiting the symptom with a formulation selected from the group consisting of Formula A, Formula B and Formula C, thereby reducing said symptom.

48. The method of claim 46 or 47, wherein any one of Formula A, Formula B or Formula C further comprises an effective amount of lutein or xanthin.

49. The method of any one of claims 38 to 48, further comprising eliminating from said subject's diet at least one of red meat, chemically-purified water, difficult to digest foodstuffs or low nutritional value foodstuffs.
